(19) United States
(12) Patent Application Publication
(10) Pub. No.: US 2025/0034066 A1
(43) Pub. Date: Jan. 30, 2025

(54) 100% HYDROGEN-FIRED LIQUID CRACKING FURNACE

(71) Applicants: Rian Reyneke, Katy, TX (US); Mark Philip Loring, Kerrville, TX (US)

(72) Inventors: Rian Reyneke, Katy, TX (US); Mark Philip Loring, Kerrville, TX (US)

(73) Assignee: KELLOGG BROWN & ROOT LLC, Houston, TX (US)

(21) Appl. No.: 18/786,224

(22) Filed: Jul. 26, 2024

Related U.S. Application Data
(60) Provisional application No. 63/516,066, filed on Jul. 27, 2023.

(51) Int. Cl. C07C 11/04; C07C 4/04

(52) U.S. Cl. CPC C07C 11/04; C07C 4/04

(57) ABSTRACT

A method and apparatus for high-purity hydrogen fired liquid hydrocarbon cracking. In one embodiment, the method may include preheating a liquid hydrocarbon feedstock in a first heat exchanger that is external to a furnace, adding steam to the preheated liquid hydrocarbon feedstock to create a fully or substantially fully vaporized stream, heating the fully or substantially fully vaporized stream in a second heat exchanger that is external to the furnace, and cracking the heated fully or substantially fully vaporized stream after it is preheated in the furnace to create a cracked gas stream comprising ethylene and hydrogen.

This application claims the benefit of U.S. Provisional Application No. 63/516,066, filed Jul. 27, 2023, which is incorporated herein by reference in its entirety.

### TECHNICAL FIELD

The present disclosure relates to methods and systems for cracking liquid hydrocarbons using pure or near pure hydrogen as a fuel gas BACKGROUND

Ethylene is manufactured in great amounts. Ethylene may be produced using a process called “steam cracking,” which may be a thermal process where hydrocarbons can be broken down, or “cracked” into smaller molecules that may then be used to manufacture more useful (and valuable) chemicals such as ethylene. In the petrochemical industry, two feedstocks for steam crackers include naphtha and ethane. Naphtha may be commonly in liquid form and primarily derived from crude oil. Steam cracking these hydrocarbons may be accomplished by first mixing them with steam, then running them through tubes in a cracking furnace where the feedstock is briefly heated. The cracked feedstock may then be rapidly quenched (cooled) to stop the hydrocarbon molecules from being completely consumed. The resulting cracked streams may be separated and purified, leaving valuable olefins such as ethylene.

Steam cracking may be very energy intensive and may contribute substantially to global CO2 emissions depending on the fuel gas burned by the cracking furnaces. Fuel gas used in liquid feed (e.g., liquid naphtha) steam cracking furnaces may contain 80-85 mol % methane and 15-20 mol % hydrogen. While combustion of hydrogen has no associated CO2 emission, combustion of methane produces approximately 230 kg CO2/Gcal of fired duty.

### SUMMARY

Provided herein are systems and methods to address these shortcomings of the art and provide other additional or alternative advantages.

In examples, a method includes preheating a stream of combustion air to yield a preheated combustion air stream, mixing a high purity hydrogen stream with the preheated combustion air stream to create a combustion mixture, burning the combustion mixture in a furnace, preheating a liquid hydrocarbon feedstock in a first heat exchanger that is external to the furnace, adding steam to the preheated liquid hydrocarbon feedstock to create a vaporized stream, preheating the vaporized stream in a second heat exchanger that is external to the furnace, and cracking the preheated vaporized stream after it is preheated in the furnace to create a cracked gas stream comprising ethylene and hydrogen. The stream of combustion air may be heated in a third heat exchanger that is internal to the furnace. The preheated vaporized stream may be further heated before it is cracked in the second heat exchanger using heat of the cracked gas stream. The high purity hydrogen stream may comprise at least 95 mol % hydrogen, for example >98 mol % hydrogen, for example about 98.5 mol % or more hydrogen, for example about 99 mol % or more hydrogen, for example about 99.5 mol % or more hydrogen, for example about 100 mol % hydrogen. The liquid hydrocarbon feedstock may be heated in the first heat exchanger to a partially vaporized state. The liquid hydrocarbon feedstock may comprise liquid naphtha. The steam may be heated by a device positioned external to the furnace before the steam is added to the stream of preheated liquid hydrocarbon feedstock. The quenching the cracked gas stream may create a quenched stream, and quenched stream may be used to preheat the vaporized stream in the second heat exchanger.

In examples, an apparatus includes a furnace configured to burn a mix of preheated air and high purity hydrogen fuel gas, a first heat exchanger positioned external to the furnace and configured to preheat a liquid hydrocarbon feedstock, a second heat exchanger positioned external to the furnace and configured to heat and vaporize a mix of steam and the preheated liquid hydrogen feedstock to create a vaporized stream, a third heat exchanger positioned internal to the furnace and configured to preheat the vaporized stream, wherein the furnace is configured to crack the preheated vaporized stream to create a cracked gas stream. The apparatus may further include a fourth heat exchanger positioned internal to the furnace and configured to preheat ambient air to yield the preheated air. The high purity hydrogen fuel gas may comprise at least 95 mol % hydrogen, for example >98 mol % hydrogen, for example about 98.5 mol % or more hydrogen, for example about 99 mol % or more hydrogen, for example about 99.5 mol % or more hydrogen, for example about 100 mol % hydrogen. The first heat exchanger may be configured to preheat the liquid hydrocarbon feedstock to a partially vaporized state. The liquid hydrocarbon feedstock may comprise liquid naphtha. The apparatus may further include a device positioned external to the furnace for heating the steam before the steam is added to the stream of preheated liquid hydrocarbon feedstock. The furnace may comprise a tube configured for receiving and cracking the vaporized stream after it is preheated. The apparatus may further include a quench exchanger for cooling the cracked gas stream to create a quenched stream. The second heat exchanger may be configured to use heat of the quenched stream to preheat the vaporized stream. The apparatus may further include a first separator for separating the cracked gas stream into a stream comprising ethylene and the stream of high purity hydrogen.

In examples, a method may include preheating a stream of combustion air to yield a preheated combustion air stream, mixing a high purity hydrogen stream with the preheated combustion air stream to create a combustion mixture, burning the combustion mixture in a furnace, preheating a liquid hydrocarbon feedstock in a heat exchanger that is external to the furnace, wherein the liquid hydrocarbon feedstock is preheated to a temperature that is dependent on a mol % H2 in the high purity hydrogen stream. The method may further include adding steam to the preheated liquid hydrocarbon feedstock to create a fully or substantially fully vaporized stream, preheating the fully or substantially fully vaporized stream in a second heat exchanger that is external to the furnace, and cracking the fully or substantially fully vaporized stream after it is preheated in the furnace to create a cracked gas stream comprising ethylene and hydrogen

In examples, a method may include preheating a liquid hydrocarbon feedstock in a first heat exchanger that is external to a furnace, adding steam to the preheated liquid hydrocarbon feedstock to create a fully or substantially fully vaporized stream, heating the fully or substantially fully vaporized stream in a second heat exchanger that is external to the furnace, and cracking the heated fully or substantially fully vaporized stream after it is preheated in the furnace to create a cracked gas stream comprising ethylene and hydrogen. The method may further include preheating a stream of combustion air to yield a preheated combustion air stream, mixing a high purity hydrogen stream with the preheated combustion air stream to create a combustion mixture, burning the combustion mixture in the furnace, wherein the liquid hydrocarbon feedstock is preheated to a temperature that is dependent on a mol % H2 in the high purity hydrogen stream.

### BRIEF DESCRIPTION OF THE DRAWINGS

The present disclosure is illustrated by way of example, and not by way of limitation, in the figures of the accompanying drawings in which like reference numerals refer to similar elements.

FIG. 1 is a schematic diagram illustrating relevant aspects of an example steam-cracking furnace.

FIG. 2 is a schematic diagram illustrating relevant aspects of an example hydrogen recovery system.

FIG. 3 is a schematic diagram illustrating relevant aspects of a steam-cracking furnace according to an example of the present disclosure.

### DETAILED DESCRIPTION

The following description sets forth numerous specific details such as examples of specific systems, components, methods, and so forth, to provide a good understanding of various examples of the techniques described herein.

However, it will be apparent to one skilled in the art that at least some examples may be practiced without these specific details. In other instances, well-known components, elements, or methods are not described in detail or are presented in a simple block diagram format to avoid unnecessarily obscuring the techniques described herein. Thus, the specific details set forth hereinafter are merely exemplary. Particular implementations may vary from these exemplary details and still be contemplated to be within the spirit and scope of the present disclosure.

Reference in the description to “an example,” “one example,” “some examples,” and “various examples” means that a particular feature, structure, step, operation, or characteristic described in connection with the example(s) may be included in at least one example of the disclosure. Further, the appearances of the phrases “an example,” “one example,” “some examples,” and “various examples” in various places in the description do not necessarily all refer to the same example(s).

The description includes references to the accompanying drawings, which form a part of the detailed description. The drawings show illustrations in accordance with examples. These examples are described in enough detail to enable those skilled in the art to practice the claimed subject matter described herein. The examples may be combined, other examples may be utilized, or structural, logical, and electrical changes may be made without departing from the scope and spirit of the claimed subject matter. It should be understood that the examples described herein are not intended to limit the scope of the subject matter but rather to enable one skilled in the art to practice, make, and/or use the subject matter.

Steam cracking of liquid hydrocarbon feedstock (e.g., light naphtha) may be very energy intensive and may contribute to global CO2 emissions. In examples, fuel gas containing a mix of hydrocarbons (e.g., methane) and hydrogen may be burned by steam-cracking furnaces to provide the heat for net process heating, to satisfy the cracking heat of reaction (endothermic reaction) and generate steam. Fuel gas used in liquid feed steam-cracking furnaces (hereinafter furnaces) typically contain 80-85 mol % methane and 15-20 mol % hydrogen. While combustion of hydrogen has no associated CO2 emission, combustion of methane produces substantial CO2/Gcal of fired duty.

It may be possible to substantially reduce or eliminate CO2 emissions from a furnace by using high purity hydrogen as fuel gas. For purposes of this description, high purity hydrogen refers to a stream that includes greater than 95 mol % H2, or greater than 97 mol % H2, or greater than 98 mol % H2. In examples, a high purity hydrogen stream may refer to a stream that includes about 98.5 mol % H2 or more, for example about 99 mol % H2 or more, for example about 99.5 mol % hydrogen or more, for example about 100 mol % H2.

However, hydrogen as a fuel gas presents problems. Hydrogen is expensive, so a furnace designed to burn high purity hydrogen as fuel should be designed to minimize the required fired duty. Compared to conventional methane-rich fuel gas, significantly less combustion air may be burned high purity hydrogen. The reduction of combustion air may alter the heat balance in the convection section of the furnace and make it more difficult to satisfy feed preheat requirements. The reduction of combustion air may also adversely impact additional features of the furnace such as a reduction of steam generation. Further, specialized furnace burners may be required in furnaces to handle high purity hydrogen fuel.

In examples, the method and system as described may provide a low or zero carbon emission system.

In examples, according to the process and system described herein may include the use of high purity hydrogen as fuel gas to the furnace. In examples, the high purity hydrogen may be the only fuel gas for the furnace that is mixed with combustion air.

In examples, the process and system described can address a duty energy balance in the system to enable the use of high purity hydrogen as the only fuel gas for the furnace to mix with combustion air.

In examples, the method and system may include the application of a combustion air preheat. In examples, the air preheat may be carried out using heat from the flue gas. In examples, the air may be preheated to 400-450° C. before it is mixed with the high purity hydrogen fuel gas.

The combination of air preheat and high purity hydrogen firing may substantially reduce available heat in the flue gas, which in turn may require changes to how the feed is heated when the flue gas is used as a heat source. In examples, the mol % H2 in the high purity hydrogen may determine how the feed is heated. For example, if the mol % H2 is 95%, a lower temperature feed may be used, and if the mol % H2 is 99.5%, a higher temperature feed may be used. In examples, the system and method may include reducing heat recovery in Primary Quench Exchangers (PQEs) to shift more duty into a feed to effluent exchanger. In examples, the feed may be partially vaporized outside the furnace using steam or another suitable heat source. In examples, superheated dilution steam can be added to the partially vaporized feed to fully vaporize the feed, which may result in a fully vaporized mixed feed stream. In examples, the fully vaporized mixed feed stream can be superheated in a feed to effluent heat exchanger using the furnace effluent leaving the PQEs.

In examples, the system and method may include a combination of two or more of the above features. In examples, the system and method may include a combination of all of the above features.

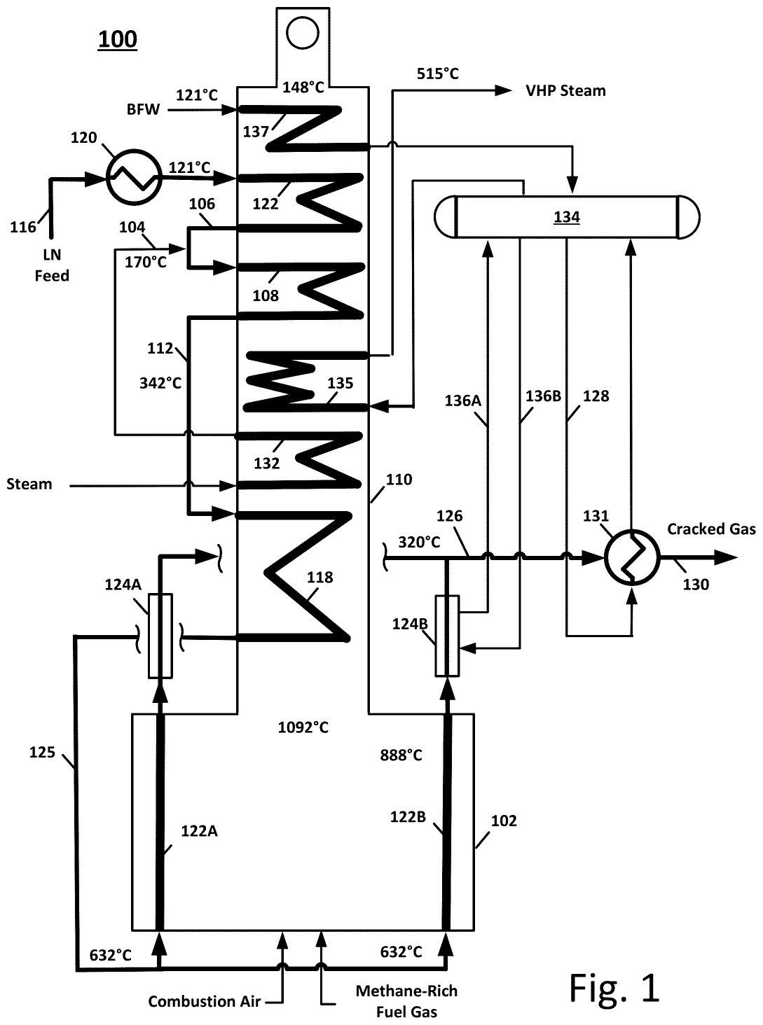

FIG. 1 is a schematic diagram illustrating a liquid feed cracking furnace system 100 that burns a methane-rich fuel gas. Furnace system 100 includes a radiant section (i.e., a fire box) 102 where a mixture of unheated combustion air and fuel gas may be burnt in burners (not shown), and a convection section 110 where heat from hot flue gas can be recovered for various processes including a process for creating superheated steam. Temperatures are shown at various locations within the furnace.

A liquid naphtha (LN) feed may enter the furnace system 100 via feed line 116. The LN feed may be preheated using a feed/effluent exchanger 120, and then partially vaporized using feed heater (e.g., heat exchanger) 122 positioned within the convection section 110. The feed heater 122 may heat the LN feed to about 170° C. Dilution steam 104 may be added to the partially vaporized stem 106, and the resulting mixture may be fully vaporized using heater 108 positioned within convection section 110. The vaporized feed 112 may be further heated by heater 118 positioned in convection section 110 before the feed is provided to the radiant section 102 via line 125. Heated, vaporized stream 125 may then passed through tube banks 122A and 122B in the radiant section 102 where the cracking reaction occurs.

In the illustrated example, flue gas in convection section 110 may be used to heat steam. For example, heater 132 positioned inside convection section 110 heats steam that may be subsequently mixed with the partially vaporized LN feed 106. Additionally, steam heated in superheater 135 produces superheated VHP steam. In examples, saturated steam may be provided to steam superheater 135 by heating boiler feed water (BFW) from steam drum 134. Quench exchangers 124A and 124B can be used to heat the BFW and provide steam, which may be subsequently superheated to provide the superheated VHP steam. In the illustrated example, BFW may be provided to quench exchangers 124A and 124B via line 136A and returned to steam drum 134 via line 136B. Similar piping to and from the quench exchanger 124A may be omitted for clarity.

Cracked gas streams from furnace tubes 122A and 122B may be cooled in primary quench exchangers 124A and 124B. The cooled cracked gas streams may then be combined in stream 126, and feed to secondary quench exchanger 131 to yield a cracked gas stream 130. Cracked gas stream 130 can be separated by a recovery section into ethylene and tail gas that includes hydrogen and CH4. The tail gas can be used as a source for fuel. Since the tail gas may contain CH4, any burn of that tail gas in a furnace would generate CO2. However, the tail gas can be separated to yield a stream of high purity H2.

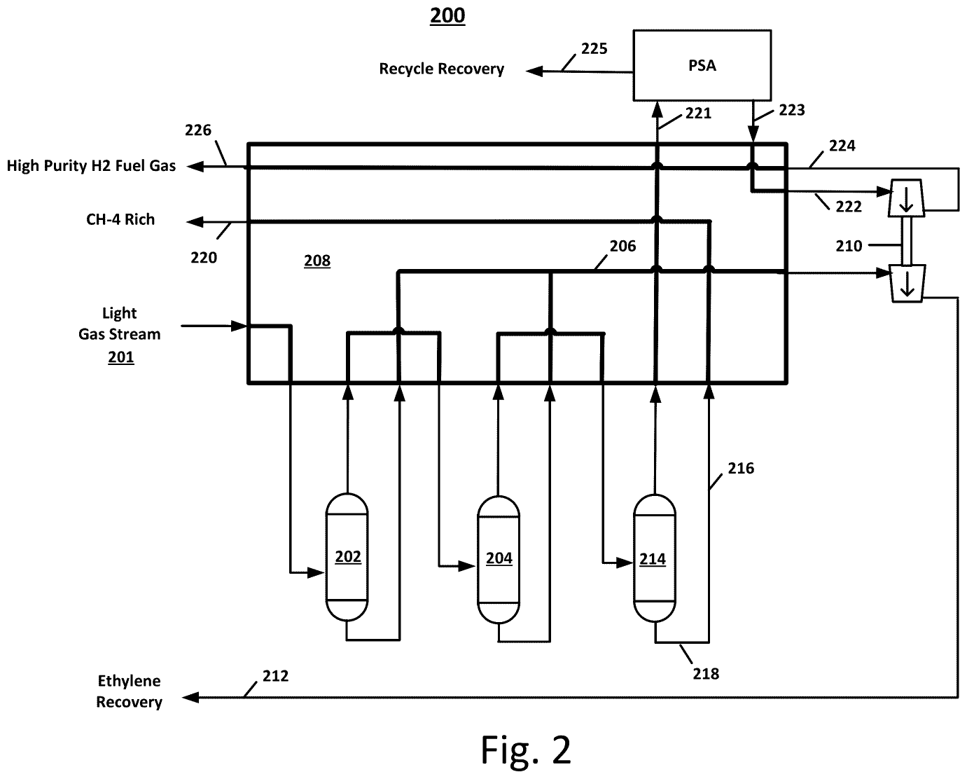

FIG. 2 is a schematic diagram illustrating some components of an example recovery section 200 that could be used for separating cracked gas 130. In examples, the cracked gas stream 130 from furnace system 100 may include H2, CH4, and ethylene. In examples, the cracked gas stream 130 may be cooled in a cold box 208. It should be noted the temperatures and pressures in this description are only illustrative of an example of the process. Depending on design constraints and considerations, the temperatures/pressures may vary, as will be appreciated by a person of skill in the art.

In examples, stream 130 may be separated into light gas stream 201 that contains H2, CH4 and some of the ethylene, and streams containing the ethylene and heavier product streams. In examples, cracked gas stream 201 is progressively cooled, and separators (e.g., knock-out drums) 202 and 204 may be used to remove ethylene. In examples, the bottom streams of the knockout drums 202 and 204, which are enriched in ethylene, can be combined into an ethylene-rich stream 206. In examples, the ethylene-rich stream 206 can be recompressed using a turbo expander/compressor 210 to provide an ethylene-rich ethylene recovery stream 212.

In examples, the top streams of the knock-out drum 204, may be enriched in CH4 and H2 and can be further cooled in the cold box and provided to a third knock-out drum 214. The temperature of the third knock-out drum 214 may be about −163° C.±10° C. In examples, the top stream 216 from the third knock-out drum 214 may include an enriched H2 stream. The bottom stream 218 may include an enriched CH4 stream. In examples, the CH4-enriched bottom stream 218 may be reheated in the cold box and exits the system as a CH4-rich stream 220.

In examples, by controlling the temperature of the third knock-out drum 214 it may be possible to determine how much CH4 is knocked out. In examples, by determining how much CH4 is knocked out it may be possible to control the purity of the H2-enriched stream.

In examples, the H2-enriched top stream 216 may be reheated in the cold box to provide H2-enriched stream 221. Stream 221 may be separated into a high purity hydrogen stream 223 and reject or recycle recovery stream 225. Reject stream 225 may be recycled for recovery of contained hydrogen.

High purity hydrogen stream 223 may be cooled in cold box to provide a cooled high purity hydrogen stream 222. According to some examples, the temperature of the cooled high purity hydrogen stream 222 can be about −140° C.±10° C. and its pressure can be about 20 to about 35 barg. In examples, cooled high purity hydrogen stream 222 may be expanded using the turbo expander/compressor 210 to yield an expanded high purity hydrogen stream 224. This drops the temperature and pressure of the expanded high purity hydrogen stream 224. For example, according to some examples, the temperature of expanded high purity hydrogen stream 224 may be about −177° C.±10° C. and the pressure may be less than about 10 barg, for example about 6 barg. The expanded high purity hydrogen stream 224 may then be reintroduced to the cold box 208, thereby providing a cold stream within the cold box capable of providing an adequate temperature approach to effect the separation of CH4 and H2 in the knockout drum 214.

In examples, the expanded high purity hydrogen stream 224 may ultimately exit the cold box as recovered high purity hydrogen stream 226 and can be sent back to a furnace system such as furnace system 300 described below as supplement high purity hydrogen fuel for the burners thereof.

In examples, the use of the recovered high purity hydrogen stream as fuel for a furnace system like furnace system 300 may not provide the sufficient fired duty for the furnace system. For example, at 100% recovery, the recovered high purity hydrogen stream may not be sufficient to meet the required fired duty for a furnace system like furnace 300 even when maximum combustion air preheating against flue gas is applied. Accordingly, in examples, the use of recovered high purity hydrogen stream, while beneficial, additional import of hydrogen to cover the fired duty shortfall may be required. Hydrogen importation can be expensive or unavailable in some locations. As such, it is advantageous to have a system and method that may be capable of balancing the fired duty to avoid importation of hydrogen.

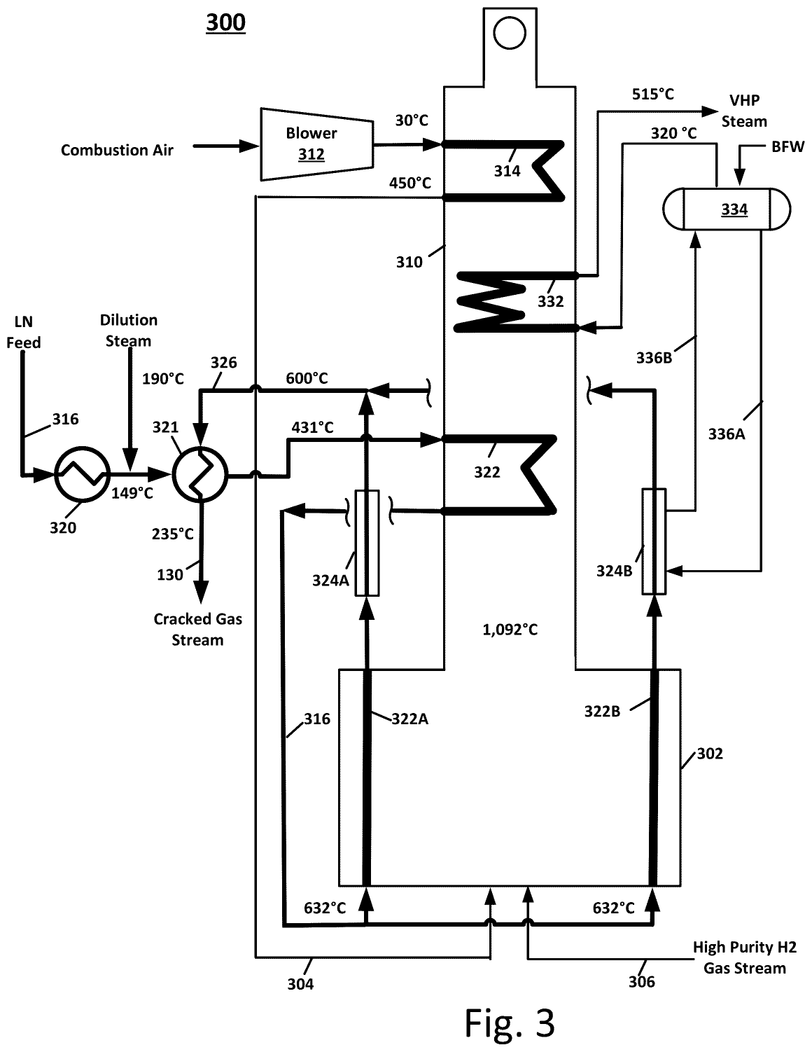

FIG. 3 is a schematic diagram illustrating relevant components of an example furnace system 300. In examples, furnace system 300 may be configured for net-zero or near net-zero steam-cracking of a liquid naphtha LN feed it being understood that other liquid feeds can be used. In examples, net zero or near net zero can be achieved by using high purity hydrogen fuel gas. However, compared to methane-rich fuel gas, a high purity hydrogen fuel gas may require significantly less combustion air to burn, which may adversely alter the heat balance in the convection section of a furnace and makes it more difficult for the convection section to satisfy LN feed vaporization and preheat requirements. The reduction of combustion air may also reduce the ability of a furnace to perform other functions such as steam generation. In examples, furnace system 300 can address these issues by preheating combustion air, externally vaporizing and superheating the feed, etc.

In examples, furnace system 300 includes a radiant section (i.e., a fire box) 302 where a mixture of preheated combustion air provided by line 304 and high purity hydrogen fuel gas provided by line 306 may be burnt in burners. In examples, some of the high purity hydrogen fuel gas may be provided by a recovery section such as recovery section 200. However, at 100% recovery, hydrogen recovered in a recovery section from cracked liquid naphtha LN feed may not be sufficient in quantity to meet the required fired duty for furnace system 300 even when the combustion air is preheated. In examples, additional import of high purity hydrogen to furnace system 300 may be necessary.

In embodiments, burner components in radiant section 302 may be made of metals that are compatible with high purity hydrogen gas. In examples, the choice of metal may depend on the design parameters of the burner, such as the operating temperature, pressure, and composition of the high purity hydrogen fuel. In examples, high purity hydrogen can cause embrittlement in some metals, so burner components may be made from metals that can be compatible with high purity hydrogen to ensure safe and reliable operation. In examples, pure nickel and nickel-based alloys may be used for hydrogen burner components because they have good resistance to hydrogen embrittlement. In examples, stainless steel may be used for hydrogen burner components since stainless steel offers good resistance to corrosion and can withstand high temperatures.

In examples, furnace system 300 may include a convection section 110 where heat from hot flue gas can be recovered for various processes. In examples, the heat recovery may be by one or more of a process for creating steam, a process for heating combustion air, a process for heating a vaporized mixture of steam and feed, etc. In examples, the temperature of the flue gas may decrease as it is sequentially used in various processes that exchange heat.

In examples, furnace system 300 may preheat the combustion air. In examples, combustion air may provide oxygen for the combustion of the high purity hydrogen fuel. In examples, by burning of the combustion air mixed with hydrogen it may be possible to generate the heat for the cracking reaction. In examples, ambient air may preheated and used for combustion. In examples, combustion air may be free or substantially free (i.e., no more than trace amounts) of methane and other like hydrocarbons that create CO2 when burned.

In examples, a proper ratio of combustion air to high purity hydrogen fuel may be used to achieve complete reaction of the LN feed, minimize formation of undesirable by-products, maximize heat output, or any combination thereof. In examples, a blower 312 may be provided to control the flow of combustion air to furnace system 300 to achieve a proper percentage excess combustion air to ensure full combustion of the high purity hydrogen fuel. In examples, the percentage excess combustion air to ensure full combustion of hydrogen fuel can range from about 10 to about 15

In examples, the combustion air provided by a blower 312 may be heated in air preheater (e.g., a heat exchanger) 314 to about 425° C. (e.g., 400-450° C.). In examples, flue gas in convection section 310 may be still hot when it reaches preheater 314. In examples, by preheating the combustion air in preheater 314 using the hot flue gases, heat recovery can be achieved. In examples, this process can capture some of the waste heat to increase the temperature of the combustion air. In examples, preheating combustion air may improve the energy efficiency of furnace system 300. In examples, by raising the temperature of the combustion air, less additional heat may be required to reach the desired furnace operating temperature, which in turn may reduce high purity hydrogen fuel consumption and energy costs since high purity hydrogen fuel gas is expensive.

In examples, the LN feed may be fed to the furnace system 300 via a feed line 316. LN feed may include a mixture of hydrocarbons. In examples, to break down the hydrocarbons into simpler molecules like ethylene, the LN feed may be diluted with steam. In examples, the LN feed may be partially or fully vaporized before it may be brought to a high reaction temperature. In examples, preheating the LN feed before it enters furnace system 300 may reduce the energy to heat the LN feed to high temperatures required for cracking. In examples, preheating the LN feed may reduce the consumption of high purity hydrogen fuel and lower furnace operating costs.

In examples, the LN feed may be preheated and partially vaporized using a first feed/effluent exchanger 320 against steam or other suitable heat. In examples, if the steam or other suitable heat is provided to feed/effluent exchanger 320 for partially vaporizing the LN feed by a source external to furnace system 300, the consumption of high purity hydrogen fuel by furnace 300 may be reduced, which in turn may reduce operating costs. In examples, exchanger 320 may be positioned external to the convection section 310.

In examples, heated dilution steam may be added to the partially vaporized LN feed output of first feed/effluent exchanger 320 to create a fully or substantially fully vaporized feed stream. In examples, the heated dilution steam can be provided by a source external to furnace system 300. In examples, by using an external source of heated dilution steam it may be possible to reduce the consumption of high purity hydrogen fuel by furnace 300. In examples, the external source of heated dilution steam may include an electric heater for heating steam to produce the heated dilution steam, and the electric heater may be powered by electricity from a renewable energy source such as a wind turbine farm.

In examples, the fully or substantially fully vaporized feed stream can be superheated to create a superheated feed stream. In examples, the fully or substantially fully vaporized feed stream can be superheated by second feed/effluent exchanger 321. In examples, the fully or substantially fully vaporized feed steam can be superheated to about 431° C. (e.g., 420-440° C.). In examples, the superheating may be done against furnace effluent leaving PQEs 324A and 324B. In examples, an exchanger 321 may be positioned external to the convection section 310. In examples, heat recovered by PQEs 324A and 324B and used by the second feed/effluent exchanger 321, can be adjusted. In examples, more heat can be shifted from PQEs 324A and 324B to second feed/effluent exchanger 321 to superheat the fully or substantially fully vaporized feed stream. In examples, this may reduce the consumption of high purity hydrogen fuel by furnace 300. In examples, this in turn may reduce operating costs.

In examples, the superheated feed stream from second feed/effluent exchanger 321 may be preheated using preheater (e.g., heat exchanger) 322 to create a preheated feed stream. In examples, preheater 322 exchanges heat with hot flue gas in convection section 310. In examples, the feed preheater 322 may preheat the superheated feed stream to about 632° C. (e.g., 620-640° C.). In examples, preheating the superheated feed stream in preheater 322 may reduce the consumption of expensive high purity hydrogen fuel by furnace 300.

In examples, the preheated feed stream from preheater 322 may be passed through the radiant section 302 of furnace 300 via tube banks 322A and 322B where the cracking reaction can occur to create cracked gas streams. In examples, two tube banks 322A and 322B are illustrated in the drawing, but it should be appreciated that one or more tubes may be used.

In examples, cracked gas streams output by tube banks 322A and 322B may be cooled to about 600° C. (e.g., 580-620° C.) in PQEs 324A and 324B. In examples, the cooled cracked gas streams may then be combined in stream 326. In examples, the combined stream 326 may be used by feed/effluent exchanger 321 to superheat the fully vaporized feed stream. In examples, after passing feed/effluent exchanger 321 the combined stream 326 exits furnace system 300 as cracked gas stream 130, which can be processed in recovery section 200 to extract ethylene and high purity hydrogen gas 226. In examples, the recovered hydrogen in high purity hydrogen gas 226 may not be sufficient in quantity for the requirements of furnace system 300, and as a result additional high purity hydrogen may be imported to furnace system 300.

The hot flue gas may be used as a source of heat in convection section 310. In examples, hot flue gas in convection section 310 may be used to preheat the combustion air via air preheater 314 and to preheat the superheated feed stream via preheater 322. In addition, the hot flue gas in convection section 310 can be used for other purposes. In examples, PQEs 324A and 324B can be used to heat BFW from steam drum 334 to create saturated steam. In examples, BFW may be provided to the PQEs 324A and 324B via line 336A and saturated steam returns to the steam drum 334A via line 336B. Similar piping to and from the PQE 324A is omitted for clarity. In examples, saturated steam may be provided to steam superheater 332 from steam drum 334. In examples, the flue gas may be used to heat saturated steam in a steam superheater 332 to produce superheated VHP steam.

In examples, furnace system 300 may be able to approach zero scope 1 (direct) CO2 emissions as defined by the Greenhouse Gas Protocol. In examples, to achieve net zero CO2 emissions overall for furnace system 300 (when also considering scope 2 emissions associated with steam and power import), electric power used for any electrification of recovery equipment to compensate for a reduction in steam generation may have to come from renewable energy sources such as wind turbine farms that lack associated CO2 emissions. In examples, furnace system 300 can provide a capital and energy-efficient way to achieve net zero carbon emissions for liquid cracking while minimizing the amount of high purity hydrogen required as fuel.

### Experimental Data

The table below compares example test results obtained for a conventional base case (Base) furnace design, a conventional furnace design (Case 1) like that shown in FIG. 1 using 100 mol % hydrogen fuel gas, and a furnace design (Case 2) like that shown in FIG. 2 using 100 mol % hydrogen fuel gas. The evaluation was based on a furnace that cracks light naphtha at a propylene to ethylene ratio of 0.54 and steam/hydrocarbon ratio of 0.5.

| Description | UOM | Base | Case 1 | Case 2 |
|---|---|---|---|---|
| Hydrocarbon Feed | kg/hr | 62406 | 62406 | 62406 |
| Dilution Steam | kg/hr | 31203 | 31203 | 31203 |
| SHP steam generated | kg/hr | 76174 | 76229 | 40575 |
| Fuel gas flow | kg/hr | 8803 | 3803 | 2720 |
| Flue gas flow | kg/hr | 175551 | 166280 | 104951 |
| Case 2/Case 1% | | | | 72% |
| Hydrogen use | | | | |
| Primary Quench | C. | 361.0 | 361.0 | 600.0 |
| (PQE) Outlet | | | | |
| Secondary Quench | C. | 343.0 | 343.0 | 343.0 |
| (SQE) Outlet | | | | |
| Air preheat temperature | C. | | | 450.0 |
| Fired Duty | MMkcal/hr | 107.53 | 108.92 | 77.90 |
| Fuel gas Composition | | | | |
| Hydrogen in fuel gas | Mol % | 16.91% | 100.00% | 100.00% |
| Methane in fuel gas | Mol % | 79.23% | 0.00% | 0.00% |
| CO in fuel gas | Mol % | 0.13% | 0.00% | 0.00% |
| CO2 in Flue Gas | kg/hr | 23910.9 | 99.5 | 62.6 |
| Oxygen in flue gas | mol % | 1.70% | 3.70% | 1.70% |

Case 1 achieves near-zero CO2 emission in the furnace flue gas by applying 100% hydrogen. However, the percent excess combustion air had to be increased to maintain performance of the convection section. Case 2 shows that hydrogen consumption can be reduced to 72% of Case 1 through application of combustion air preheat and changes to the feed preheat arrangement and convection section design as described earlier. This may substantially reduce the external high purity hydrogen import.

In examples, furnace system 300 can reduce the total fuel gas (or natural gas) to produce ethylene and supplemental hydrogen, which also reduces the total amount of CO2 that to be captured and stored. In examples, steam generation may also be reduced, so partial electrification of compressors in the recovery section may be required to re-balance the furnace system 300. In examples, to achieve net zero emissions from the overall unit (considering scope 1 & 2 emissions), electric power import may come from renewable energy sources without associated CO2 emissions.

When ranges are disclosed herein, ranges from any lower limit may be combined with any upper limit to recite a range not explicitly recited, as well as ranges from any lower limit may be combined with any other lower limit to recite a range not explicitly recited, in the same way, ranges from any upper limit may be combined with any other upper limit to recite a range not explicitly recited. Additionally, reference to values stated in ranges includes each and every value within that range, even though not explicitly recited. Thus, every point or individual value may serve as its own lower or upper limit combined with any other point or individual value or any other lower or upper limit, to recite a range not explicitly recited.

It is understood that the present subject matter may be embodied in many different forms and should not be construed as being limited to the examples set forth herein. Rather, these examples are provided so that this subject matter will be thorough and complete and will convey the disclosure to those skilled in the art. Indeed, the subject matter is intended to cover alternatives, modifications, and equivalents of these examples. Furthermore, in the detailed description of the present subject matter, numerous specific details are set forth in order to provide a thorough understanding of the present subject matter. However, it will be clear to those of ordinary skill in the art that the present subject matter may be practiced without such specific details.

Aspects of the present disclosure are described herein with reference to flowchart illustrations and/or block diagrams of methods, apparatuses (systems), and computer program products according to examples of the disclosure. It will be understood that each block of the flowchart illustrations or block diagrams, and combinations of blocks in the flowchart illustrations or block diagrams, may be implemented by one or more apparatuses that create a mechanism for implementing the functions/acts specified in the block or blocks of the flowchart or block diagram.

The above description sets forth numerous specific details such as examples of specific systems, components, methods, and so forth, in order to provide a good understanding of several examples of the present disclosure. It is to be understood that the above description is intended to be illustrative and not restrictive. Many other examples will be apparent to those of skill in the art upon reading and understanding the above description. The scope of the disclosure should, therefore, be determined with reference to the appended claims, along with the full scope of equivalents to which such claims are entitled.

What is claimed is:

1. A method comprising:
preheating a stream of combustion air to yield a preheated combustion air stream;
mixing a high purity hydrogen stream with the preheated combustion air stream to create a combustion mixture;
burning the combustion mixture in a furnace;
preheating a liquid hydrocarbon feedstock in a first heat exchanger that is external to the furnace;
adding steam to the preheated liquid hydrocarbon feedstock to create a vaporized stream;
preheating the vaporized stream in a second heat exchanger that is external to the furnace;
cracking the preheated vaporized stream after it is preheated in the furnace to create a cracked gas stream comprising ethylene and hydrogen.

2. The method of claim 1, wherein the stream of combustion air is heated in a third heat exchanger that is internal to the furnace.

3. The method of claim 1, wherein the preheated vaporized stream is further heated before it is cracked in the second heat exchanger using heat of the cracked gas stream.

4. The method of claim 1, wherein the high purity hydrogen stream comprises at least 95% hydrogen.

5. The method of claim 1, wherein the liquid hydrocarbon feedstock is heated in the first heat exchanger to a partially vaporized state.

6. The method of claim 1, wherein the liquid hydrocarbon feedstock comprises liquid naphtha.

7. The method of claim 1, wherein the steam is heated by a device positioned external to the furnace before the steam is added to the stream of preheated liquid hydrocarbon feedstock.

8. The method of claim 1 further comprising quenching the cracked gas stream to create a quenched stream.

9. The method of claim 8, wherein heat of the quenched stream is used to preheat the vaporized stream in the second heat exchanger.

10. An apparatus comprising:
a furnace configured to burn a mix of preheated air and high purity hydrogen fuel gas;
a first heat exchanger positioned external to the furnace and configured to preheat a liquid hydrocarbon feedstock;
a second heat exchanger positioned external to the furnace and configured to heat and vaporize a mix of steam and the preheated liquid hydrogen feedstock to create a vaporized stream;
a third heat exchanger positioned internal to the furnace and configured to preheat the vaporized stream;
wherein the furnace is configured to crack the preheated vaporized stream to create a cracked gas stream.

11. The apparatus of claim 10 further comprising a fourth heat exchanger positioned internal to the furnace and configured to preheat ambient air to yield the preheated air.

12. The apparatus of claim 10, wherein high purity hydrogen fuel gas comprises at least 95% hydrogen.

13. The apparatus of claim 10, wherein the first heat exchanger is configured to preheat the liquid hydrocarbon feedstock to a partially vaporized state.

14. The apparatus of claim 10, wherein the liquid hydrocarbon feedstock comprises liquid naphtha.

15. The apparatus of claim 10 further comprising a device positioned external to the furnace for heating the steam before the steam is added to the stream of preheated liquid hydrocarbon feedstock.

16. The apparatus of claim 10, wherein the furnace comprises a tube configured for receiving and cracking the vaporized stream after it is preheated.

17. The apparatus of claim 16 further comprising a quench exchanger for cooling the cracked gas stream to create a quenched stream.

18. The apparatus of claim 17 wherein the second heat exchanger is configured to use heat of the quenched stream to preheat the vaporized stream.

19. The apparatus of claim 10 further comprising:
a first separator for separating the cracked gas stream into a stream comprising ethylene and the stream of high purity hydrogen.

20. The apparatus of claim 10 wherein the mix comprises methane.

21. A method comprising:
preheating a stream of combustion air to yield a preheated combustion air stream;
mixing a high purity hydrogen stream with the preheated combustion air stream to create a combustion mixture; and
burning the combustion mixture in a furnace;
preheating a liquid hydrocarbon feedstock in a heat exchanger that is external to the furnace;
wherein the liquid hydrocarbon feedstock is preheated to a temperature that is dependent on a mol % H2 in the high purity hydrogen stream.

22. The method of claim 21 further comprising:
adding steam to the preheated liquid hydrocarbon feedstock to create a fully or substantially fully vaporized stream;
preheating the fully or substantially fully vaporized stream in a second heat exchanger that is external to the furnace;
cracking the fully or substantially fully vaporized stream after it is preheated in the furnace to create a cracked gas stream comprising ethylene and hydrogen.

23. A method comprising:
preheating a liquid hydrocarbon feedstock in a first heat exchanger that is external to a furnace;
adding steam to the preheated liquid hydrocarbon feedstock to create a fully or substantially fully vaporized stream;
heating the fully or substantially fully vaporized stream in a second heat exchanger that is external to the furnace;
cracking the heated fully or substantially fully vaporized stream after it is preheated in the furnace to create a cracked gas stream comprising ethylene and hydrogen.

24. The method of claim 23 further comprising:
preheating a stream of combustion air to yield a preheated combustion air stream;
mixing a high purity hydrogen stream with the preheated combustion air stream to create a combustion mixture; and
burning the combustion mixture in the furnace;
wherein the liquid hydrocarbon feedstock is preheated to a temperature that is dependent on a mol % H2 in the high purity hydrogen stream.
